(19) United States
(12) Patent Application Publication
(10) Pub. No.: US 2007/0232680 A1
(43) Pub. Date: Oct. 4, 2007

(54) PREPARATION OF RAMIPRIL AND STABLE PHARMACEUTICAL COMPOSITIONS

(76) Inventors: Vijayabhaskar Bolugoddu, Hyderabad (IN); Srinivas Reddy Mamilla, Secunderabad (IN); Chinamala Kondaiah Golla, Hyderabad (IN); Srinivasa Reddy Madduri, Guntur (IN); Ravinder Kodipyaka, Kaghaznagar (IN); Surajit Das, Kolkata (IN); Indu Bhushan, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/696,372

(22) Filed: Apr. 4, 2007

Related U.S. Application Data
(60) Provisional application No. 60/820,183, filed on Jul. 24, 2006.
(60) Provisional application No. 60/870,007, filed on Dec. 14, 2006.

(30) Foreign Application Priority Data
Apr. 4, 2006 (IN) 612/CHE/2006
Sep. 27, 2006 (IN) 1789/CHE/2006

(51) Int. Cl. A61K 31/403; C07D 209/02

(52) U.S. Cl. 514/412; 548/452

(57) ABSTRACT

A process for preparing ramipril, and stable pharmaceutical compositions containing ramipril.

### INTRODUCTION TO THE INVENTION

The present invention relates to a process for the preparation of ramipril. In particular, the present invention relates to a process for the preparation of substantially pure ramipril and its pharmaceutically acceptable salts.

The present invention also relates to stable oral pharmaceutical formulations comprising substantially pure ramipril or its pharmaceutically acceptable salts and a stabilizing amount of magnesium oxide, processes for their preparation, and methods of treatment involving administration of such compositions.

Ramipril has a chemical name (2S,3aS,6aS)-1[(S)-N-[(S)-1-carboxy-3-phenylpropyl]alanyl]octahydrocyclopenta[b]pyrrole-2-carboxylic acid, 1-ethyl ester, and is structurally represented by Formula I.

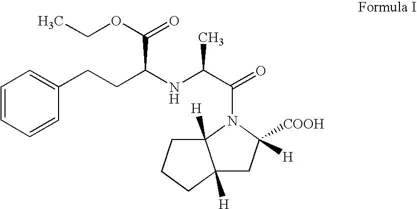

Ramiprilat, the diacid metabolite of ramipril, is a non-sulfhydryl angiotensin converting enzyme inhibitor. Ramipril is converted to ramiprilat by hepatic cleavage of the ester group in vivo and is useful as a antihypertensive agent. It is available in the market under the brand name ALTACE® as capsules for oral administration in the dosage forms of 1.25 mg, 2.5 mg, 5 mg, and 10 mg of ramipril.

U.S. Pat. No. 4,587,258 and European Patent No. 0 115 345 B1 disclose ramipril and its homologues along with their pharmaceutically acceptable salts.

Processes for the preparation of ramipril have also been described in Canadian Patent No. CA 1,338,162, European Patent No. EP 79022, U.S. Pat. Nos. 5,977,380 and 6,407,262, Tetrahedron Letters, 1993, 34(41), 6603-6606, and Heterocycles, 1989, 28(2), 957-965.

The synthesis of ramipril involves many synthetic steps in which undesired products are obtained. Therefore, the final product can be contaminated not only with the undesired products derived from the last synthetic step of the process but also with compounds that were formed in previous steps. These products should be removed from the final product in order to meet the ICH specifications for purity.

Regulatory authorities worldwide require that drug manufacturers isolate, identify and characterize the impurities in their products. Moreover, it is required to control the levels of these impurities in the final drug compound obtained by the manufacturing process and to ensure that the impurity is present in the lowest possible levels.

Hence, there is a need for a purification method for ramipril that uses a simple and commercially viable process while achieving the desired purity. Even though crystallization is known to be the simplest process that can be used for purification of organic compounds, many of the impurities are hard to remove as they co-crystallize with ramipril. The right choice of solvents for crystallization plays a major role in removing the undesired impurities from the compound and therefore purifying it. The solvent of choice should effectively remove the impurity without sacrificing the yield.

ACE inhibitors like ramipril on contact with some of the commonly used pharmaceutical excipients undergo degradation at accelerated rates due to:

i) Cyclization via internal nucleophilic attack to form substituted diketopiperazines;

ii) Hydrolysis of the side chain ester group; and

iii) Oxidation to form products having unwanted coloration.

International Application Publication Nos. WO 99/62560 and WO 03/059388, U.S. Patent Application Publication No.2003/0215526, and U.S. Pat. Nos. 4,743,450, 4,830,853 and 4,793,998, disclose stabilized compositions of ACE inhibitors. International Application Publication Nos. WO 2005/002548, WO 2004/064809, WO 03/059330, WO 2005/067887, WO 2005/041940, and U.S. Patent Application Publication No. 2006/0045911 disclose stable compositions of ramipril.

The above mentioned documents teach either addition of a stabilizer or a polymeric coating on the active ingredient to stabilize the pharmaceutical compositions of ACE inhibitors, which are susceptible to degradation. Coating the active ingredient is quite cumbersome and low yielding, moreover it requires specialized equipment. Thus, there lies a need to provide simple oral pharmaceutical formulations comprising ramipril or its pharmaceutically acceptable salt in a pharmaceutically acceptable carrier medium.

The present invention provides a process for the preparation of substantially pure ramipril and its pharmaceutically acceptable salts, which can be practiced on an industrial scale, and also can be carried out without sacrifice of overall yield based on the starting materials employed. The present invention also provides stable pharmaceutical compositions of substantially pure ramipril and its pharmaceutically acceptable salts, processes for their preparation and method of use.

### SUMMARY OF THE INVENTION

In an aspect, the present invention relates to a process for the preparation of ramipril. In particular, the present invention relates to a process for the preparation of substantially pure ramipril and its pharmaceutically acceptable salts. It also relates to stable oral pharmaceutical formulations comprising substantially pure ramipril or its pharmaceutically acceptable salts, processes for their preparation and their method of use.

One aspect of the present invention provides substantially pure ramipril and its pharmaceutically acceptable salts.

Another aspect of the present invention provides a process for the preparation of ramipril. In an embodiment, the process comprises:

a) reacting an acid salt of 2-azabicyclo [3,3,0]-octane-3-carboxylic acid benzyl ester, such as the hydrochloride of Formula II, where Ph is a phenyl group, with a suitable optically pure acid in the presence of an alkyl acetate as solvent to afford the corresponding diastereomeric salt of Formula III, where X is an acid anion group such as tartarate, dibenzoyl-L-tartarate, maleate, mandelate, or camphor sulphonate.

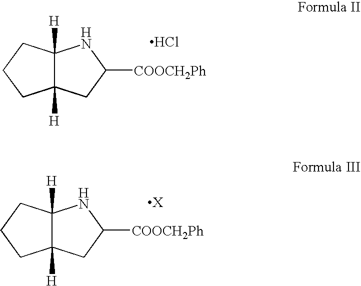

b) reacting the diastereomeric salt of Formula III with a suitable base to give the free base of S,S,S-2-azabicyclo [3,3,0]-octane-carboxylic acid benzyl ester, which can be further converted to its acid salt, such as the hydrochloride salt S,S,S-2-azabicyclo [3,3,0]-octane-carboxylic acid benzyl ester hydrochloride of Formula IV, which is optionally isolated;

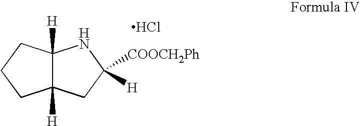

c) reacting the acid salt, such as S,S,S-2-azabicyclo [3,3,0]-octane-carboxylic acid benzyl ester hydrochloride of Formula IV, with ethoxycarbonyl phenyl propyl alanoyl chloride hydrochloride of Formula V, where Et is an ethyl group, in the presence of a suitable base to afford 1-[N-(1-(S)-carboethoxy-3-phenyl propyl)-(S)-alanyl]-cis-endo octa hydro cyclo penta[b]pyrrole-2-carboxylic acid benzyl ester of Formula VI; and

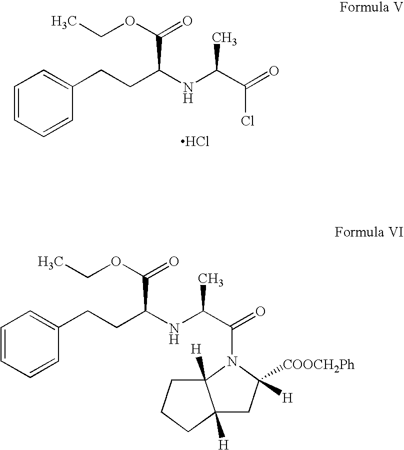

d) reacting 1-[N-(1-(S)-carboethoxy-3-phenyl propyl)-(S)-alanyl]-cis-endo octa hydro cyclo penta[b]pyrrole-2-carboxylic acid benzyl ester of Formula VI with a suitable reducing agent to afford ramipril of Formula I.

In an embodiment, a process for preparing ramipril or a salt thereof comprises:

a) reacting a compound having Formula IIa with an optically pure acid in the presence of an alkyl acetate solvent to afford a diastereomeric salt;

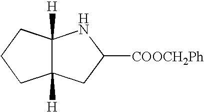

### Formula IIa

b) reacting the diastereomeric salt with a base to give a free base having Formula IV, converting a free base to its acid salt, and optionally isolating an acid salt;

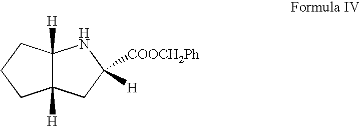

c) reacting an acid salt of b) with a compound having Formula V in the presence of a base, to afford a compound having Formula VI; and

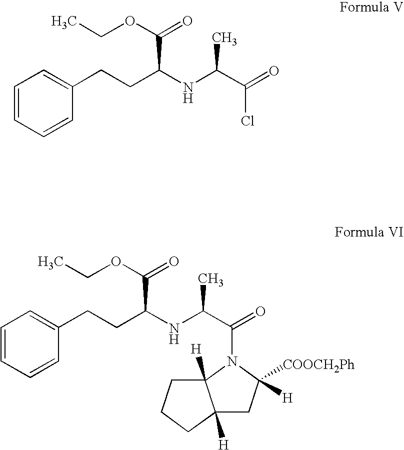

d) reacting a compound having Formula VI with a reducing agent to afford ramipril.

Yet another aspect of the present invention provides a process for the preparation of substantially pure ramipril. In an embodiment, the process comprises:

a) providing a solution of ramipril in an ether solvent, ketone solvent, water or a mixture of any two or more thereof; and

b) crystallizing a solid from the solution.

Still another aspect of the present invention is directed to compositions containing ramipril or its pharmaceutically acceptable salt, stabilized by the presence of magnesium oxide.

In one of the embodiment the invention includes the concentration of magnesium oxide used in the formulation to stabilize ramipril.

In another embodiment, a pharmaceutical composition is prepared by combining ramipril with not only a stabilizing agent comprising magnesium oxide, but also an agent that minimizes the hydrolysis of the ACE inhibitor, such as a saccharide or filler having hydrolysis-minimizing effects on ramipril.

A further aspect of the invention provides a process for preparing the stable pharmaceutical compositions of ramipril or its pharmaceutically acceptable salts.

A still further aspect of the present invention provides a method of using the pharmaceutical compositions of ramipril or its pharmaceutically acceptable salts prepared according to the process of the present invention.

An embodiment of the invention provides a method for packaging ramipril or a salt thereof, comprising:

placing ramipril in a sealed container under an inert atmosphere;

placing the sealed container, a desiccant, and an oxygen adsorbent in a second sealed container;

placing the second sealed container in a triple laminated bag and sealing; and enclosing the triple laminated bag in a closed high density polyethylene (“HDPE”) container.

A further embodiment of the invention provides a solid pharmaceutical composition comprising ramipril or a salt thereof, at least one pharmaceutical excipient, and about 0.01% to about 0.5% by weight of magnesium oxide.

### BRIEF DESCRIPTION OF THE DRAWINGS

FIG. 1 is a schematic representation of an embodiment of a process for the preparation of ramipril starting from the intermediate compound of Formula II.

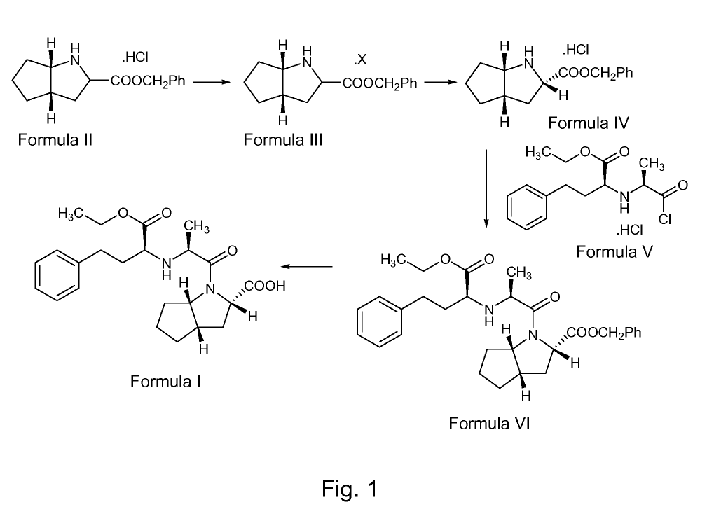

FIG. 2 is an X-ray powder diffraction pattern of crystalline ramipril prepared in Example 9.

### DETAILED DESCRIPTION OF THE INVENTION

The present invention relates to a process for the preparation of ramipril. In particular, the present invention relates to a process for the preparation of substantially pure ramipril and its pharmaceutically acceptable salts.

One aspect of the present invention provides substantially pure ramipril and its pharmaceutically acceptable salts.

By “substantially pure ramipril” it is meant that ramipril or any of the pharmaceutically acceptable salts of ramipril prepared in accordance with the present invention contains less than about 0.5%, or less than about 0.1%, by weight of any individual impurity such as ramipril methyl ester, ramipril isopropyl ester, hexahydro ramipril, ramipril diketopiperazine, ramipril diacid impurity, ECPP alanine impurity, and ramipril dimer, as characterized by a high performance liquid chromatography (“HPLC”) chromatogram obtained from a mixture comprising the desired compound and one or more of the said impurities.

The pharmaceutically acceptable salts of ramipril refer to salts prepared form pharmaceutically acceptable non-toxic bases including inorganic bases and organic bases.

Salts derived from inorganic bases include aluminium, ammonium, calcium, copper, ferric, ferrous, lithium, magnesium, manganic salts, manganous, potassium, sodium, zinc, and the like. Salts derived from organic non-toxic bases include salts of primary, secondary, tertiary amines, and substituted amines including naturally occurring substituted amines.

Ramipril or its pharmaceutically acceptable salts of the present invention are also substantially free of the isomeric impurities like the SSRRR isomer and the RRRRR isomer of ramipril. It contains less than about 0.15%, or less than about 0.1%, by weight of the SSRRR isomer and of the RRRRR isomer of ramipril.

As used herein, “ramipril methyl ester” refers to (2S, 3aS, 6aS)-1-[(S)-1-(methoxycarbonyl)-3-phenylpropyl]amino]propanoyl]octahydrocyclopenta[b]pyrrole-2-carboxylic acid represented by Formula Ia.

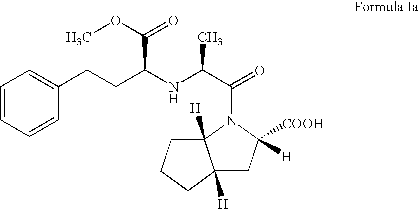

As used herein, “ramipril isopropyl ester” refers to (2S, 3aS, 6aS)-1-[(S)-1-(methoxycarbonyl)-3-phenylpropyl]amino]propanoyl]octahydrocyclopenta[b]pyrrole-2-carboxylic acid represented by Formula Ib.

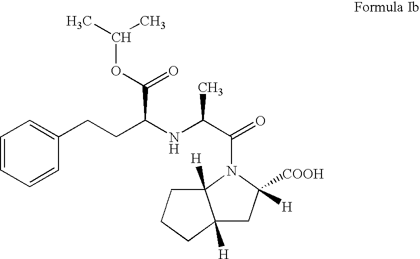

As used herein, “hexahydro ramipril” refers to (2S, 3aS, 6aS)-1-[(S)-2-[[(S)-1-(ethoxycarbonyl)-3-cyclohexyl propyl]amino]propanoyl]octahydrocyclopenta[b]pyrrole-2-carboxylic acid represented by Formula Ic.

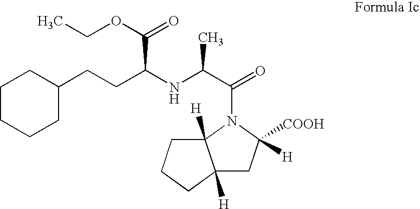

As used herein, “ramipril diketopiperazine” refers to (Ethyl (2S)-2-[3S, 5aS, 8aS, 9aS)-3-methyl-1,4-dioxodecahydro-1H-cyclopenta[e]pyrrolo[1,2-a]pyrazin-2-yl]-4-phenylbutanoate represented by Formula Id.

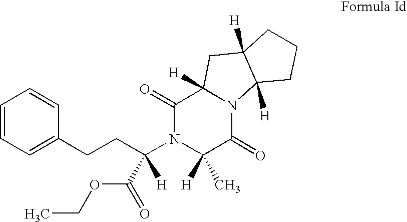

As used herein, “ramipril diacid impurity” refers to (2S, 3aS, 6aS)-1-[(S)-2-[[(S)-1-carboxy-3-phenyl propyl]-amino]propanoyl]octahydrocyclopenta[b]pyrrole-2-carboxylic acid represented by Formula Ie.

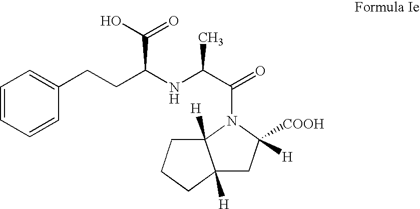

As used herein, “ECPP alanine impurity” refers to (S)-2-[[(S)-1-(ethoxycarbonyl)-3-phenylpropyl]amino]propanoic acid represented by Formula If.

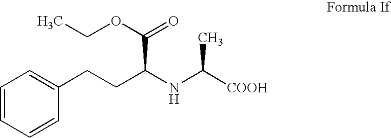

As used herein, “ramipril dimer impurity” refers to 1,4-Di(1-ethoxycarbonyl -3-phenylpropyl)3,6-dimehyl-2,5-piperzinedone”, represented by Formula Ig.

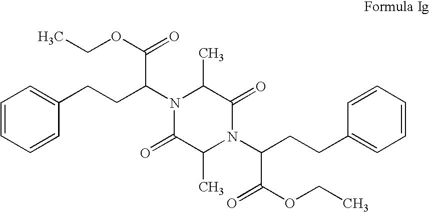

The RRRRR, and SSRRR isomers of ramipril are represented by Formula Ij and Formula In, respectively”.

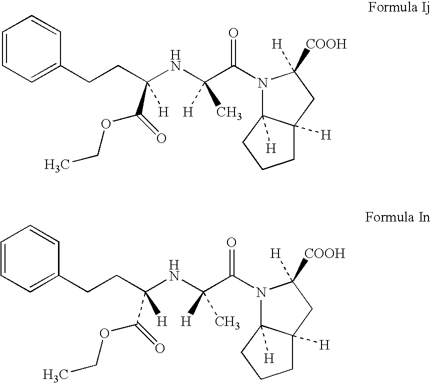

Ramipril having a reduced level of impurities typically also contains low levels of residual solvents. For purposes of the present invention, any residual solvents in purified ramipril are also considered as impurities. Residual solvents can be quantified by application of known chromatographic techniques, including gas chromatography.

Another aspect of the present invention provides a process for the preparation of ramipril. In an embodiment, the process comprises:

a) reacting an acid salt of 2-azabicyclo[3,3,0]-octane-3-carboxylic acid benzyl ester, such as the hydrochloride of Formula II, with a suitable optically pure acid, in the presence of an alkyl acetate as a solvent, to afford the corresponding diastereomeric salt of Formula III, where X is an acid anion group such as tartarate, dibenzoyl-L-tartarate, maleate, mandelate, or camphor sulphonate;

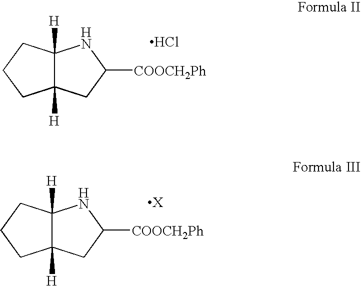

b) reacting the diastereomeric salt Formula III with a suitable base to give the free base of S,S,S-2-azabicyclo[3,3,0]-octane-carboxylic acid benzyl ester, which can be further converted to an acid salt, such as S,S,S-2-azabicyclo[3,3,0]-octane-carboxylic acid benzyl ester hydrochloride of Formula IV, which is optionally isolated;

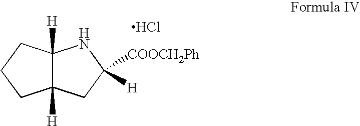

c) reacting the acid salt of S,S,S-2-azabicyclo[3,3,0]-octane-carboxylic acid benzyl ester with ethoxycarbonyl phenyl propyl alanoyl chloride hydrochloride of Formula V in presence of a suitable base to afford 1-[N-(1-(S)-carboethoxy-3-phenyl propyl)-(S)-alanyl]-cis-endo octa hydro cyclo penta[b]pyrrole-2-carboxylic acid benzyl ester of Formula VI; and

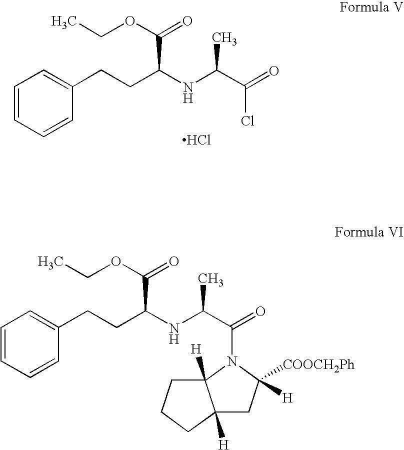

d) reacting 1-[N-(1-(S)-carboethoxy-3-phenyl propyl)-(S)-alanyl]-cis-endo octa hydro cyclo penta[b]pyrrole-2-carboxylic acid benzyl ester of Formula VI with a suitable reducing agent to afford ramipril of Formula I.

Step a) involves the reaction of an acid salt of 2-azabicyclo [3,3,0]-octane-3-carboxylic acid benzyl ester, such as the hydrochloride of Formula II, with a suitable optically pure acid, in the presence of an alkyl acetate as a solvent, to afford the corresponding diastereomeric salt of Formula III. The discussion below will describe the hydrochloride salt, although other acid salts can be used.

Suitably, the hydrochloride salt of Formula II is broken to release the free base 2-azabicyclo[3,3,0]-octane-3-carboxylic acid benzyl ester of Formula IIa before reacting with the chiral resolving agent. For example, the free base may be isolated from the salt by treatment of the salt with an aqueous base and extracting the free base into a suitable organic solvent.

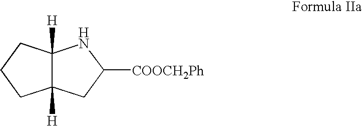

Suitable bases which can be used for breaking the acid salt include, but are not limited to: alkali metal hydrides such as lithium hydride, sodium hydride and the like; alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof. These bases can be used in the form of solids or in the form of aqueous solutions.

Suitably, aqueous solutions containing about 5% to 50%, or about 10% to 20%, (w/v) of the corresponding base can be used. Any concentration is useful, which will convert the acid addition salt to a free base.

Suitable solvents which can be used for extracting the free base include, but are not limited to: water insoluble organic solvents like ethyl acetate, n-propyl acetate, n-butyl acetate, t-butyl acetate; halogenated hydrocarbons such as dichloromethane, ethylene dichloride, chloroform, and the like; and mixtures thereof.

Suitable chiral resolving agents which can be used include, but are not limited to, tartaric acid or substituted tartaric acids such as L-tartaric acid, (−)-dibenzoyl-L-tartaric acid, maleic acid, or substituted maleic acid, mandelic acids such as L-(+)-mandelic acid, camphor sulphonic acids such as L-(+)-camphor-10-sulphonic acid, and the like.

Suitable alkyl acetates which can be used as the solvent medium for the reaction include, but are not limited to, ethyl acetate, n-propyl acetate, n-butyl acetate, t-butyl acetate, and the like, and mixtures thereof in various proportions.

Suitable temperatures for conducting the reaction range from about −10° C. to about 100° C., or from about 20° C. to about 50° C.

Step b) involves reacting the diastereomeric salt Formula III with a suitable base to give the free base S,S,S-2-azabicyclo[3,3,0]-octane-carboxylic acid benzyl ester, which can be further converted to its acid salt, such as the hydrochloride salt S,S,S-2-azabicyclo[3,3,0]-octane-carboxylic acid benzyl ester hydrochloride of Formula IV, which is optionally isolated.

Suitable bases which can be used for the displacement of acid from the diastereomeric salt include but are not limited to: hydroxides of alkali metals, such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and aqueous ammonia and ammonia as a gas; and mixtures thereof or their combinations with water in various proportions.

Suitable organic solvents which can be used for extraction of the free base of S,S,S-2-azabicyclo[3,3,0]-octane-carboxylic acid benzyl ester include but are not limited to: alcohols such as methanol, ethanol, isopropyl alcohol, n-butanol, and the like; ketones such as acetone, ethyl methyl ketone, methyl isobutyl ketone, and the like; esters such as ethyl acetate, n-propyl acetate, n-butyl acetate, t-butyl acetate, and the like; nitriles such as acetonitrile, priopionitrile, and the like; halogenated hydrocarbons such as dichloromethane, ethylene dichloride, chloroform, and the like; and mixtures thereof or their combinations with water in various proportions.

Suitably, the free base can be converted to its acid addition salt. Suitable acids which can be used for preparing the acid addition salt include, but are not limited to those that form anions like acetate, benzoate, bicarbonate, tartarate, citrate, iodide, chloride, bromide, mesylate, and the like.

In one embodiment, the acid used is hydrochloric acid and the salt obtained is S,S,S-2-azabicyclo[3,3,0]-octane-carboxylic acid benzyl ester hydrochloride of Formula IV.

The free base obtained can be suitably converted to its hydrochloride salt by reacting with hydrochloric acid. Hydrochloric acid can be used in the form of aqueous hydrochloric acid, hydrogen chloride gas, alcoholic hydrogen chloride, ethyl acetate hydrochloride, and the like. The pH of the reaction mass during conversion to a hydrochloride salt can be adjusted to about 0.1 to about 7, or from about 0.1 to about 2.

The product can be directly progressed to the next stage without isolation, giving rise to an in-situ process.

Step c) involves the reaction of the acid salt of S,S,S-2-azabicyclo[3,3,0]-octane-carboxylic acid benzyl ester, such as the hydrochloride of Formula IV, with ethoxycarbonyl phenyl propyl alanoyl chloride hydrochloride of Formula V in the presence of a suitable base, to afford 1-[N-(1-(S)-carboethoxy-3-phenyl propyl)-(S)-alanyl]-cis-endo octa hydro cyclo penta[b]pyrrole-2-carboxylic acid benzyl ester of Formula VI.

Suitable organic solvents which can be used include but are not limited to: ketones such as acetone, ethyl methyl ketone, methyl isobutyl ketone, and the like; esters such as ethyl acetate, n-propyl acetate, n-butyl acetate, t-butyl acetate, and the like; nitriles such as acetonitrile, priopionitrile, and the like; halogenated hydrocarbons such as dichloromethane, ethylene dichloride, chloroform, and the like; and mixtures thereof or their combinations with water in various proportions.

Suitable bases which can be used for the reaction include, but are not limited to, organic bases such as pyridine, triethylamine, ethylamine, dicyclohexylamine, diisopropyl ethylamine, and the like and mixtures thereof.

Suitable temperatures for conducting the reaction range from −10° C. to about 50° C., or from about 0° C. to about 20° C.

Step d) involves reacting 1-[N-(1-(S)-carboethoxy-3-phenyl propyl)-(S)-alanyl]-cis-endo octa hydro cyclo penta[b]pyrrole-2-carboxylic acid benzyl ester of Formula VI with a suitable reducing agent to afford ramipril of Formula I.

Suitable reducing agents which can be used include but are not limited to Raney nickel, palladium on carbon, platinum dioxide and the like, in the presence of hydrogen gas.

The hydrogen pressure for the reaction can range from about 2 to about 5 kg/cm² and the temperature for conducting the reaction can range from about −10° C. to about 50° C., or from about 0° C. to about 30° C.

Yet another aspect of the present invention provides a process for the preparation of substantially pure ramipril. In an embodiment, the process comprises:

a) providing a solution of ramipril in an ether solvent, a ketone solvent, water or any mixtures of two or more thereof; and

b) isolating a solid from the solution.

Step a) involves providing a solution of ramipril in an ether solvent, ketone solvent, water or any mixtures thereof.

Ramipril for the purpose of purification may be prepared according to any process, including a process described in the art, or using a process similar to the one described above.

The solution of ramipril may be obtained by dissolving ramipril in a solvent, or a solution may be obtained directly from a reaction in which ramipril is formed.

When the solution is prepared by dissolving ramipril in a solvent, any form of ramipril such as any crystalline or amorphous form including any salts, solvates and hydrates may be utilized for preparing the solution.

Suitable solvents which can be used for dissolving ramipril include but are not limited to: ethers such as dimethyl ether, diethyl ether, diisopropyl ether, methyl tertiary-butyl ether, tetrahydrofuran, 1-4-dioxane, and the like; ketones such as acetone, ethyl methyl ketone, methyl isobutyl ketone and the like; water; and mixtures thereof in various proportions.

The dissolution temperatures can range from about 20 to 120° C. depending on the solvent used for dissolution. Any other temperature is also acceptable as long as the stability of ramipril is not compromised and a clear solution is obtained.

The quantity of solvent used for dissolution depends on the solvent and the dissolution temperature adopted. The concentration of ramipril in the solution may generally range from about 0.1 to about 10 g/ml in the solvent. Solutions can optionally be concentrated by evaporating excess solvent, since concentrated solutions generally will afford a higher yield of product.

The solution obtained can be optionally treated with activated charcoal to enhance the color of the compound followed by filtration through a medium such as through a flux calcined diatomaceous earth (Hyflow) bed to remove the carbon.

The carbon treatment can be conducted either at the dissolution or concentration temperatures, or after cooling the solution to lower temperatures.

Step b) involves isolation of a solid from the solution.

For isolation to occur, the mass may be maintained at temperatures lower than the concentration or solution formation temperatures, such as, for example, below about 10° C. or about 25° C., for a period of time as required for a more complete isolation of the product. The exact cooling temperature and time required for complete isolation can be readily determined by a person skilled in the art and will also depend on parameters such as concentration and temperature of the solution or slurry.

Optionally isolation may be enhanced by methods such as cooling, partial removal of the solvent from the mixture, by adding an anti-solvent to the reaction mixture, or a combination thereof.

The solid material is recovered from the final mixture, with or without cooling below the operating temperature, using any technique such as filtration by gravity or by suction, decantation, centrifugation, and the like. The crystals so isolated can carry a small proportion of occluded mother liquor containing a higher percentage of impurities. If desired the crystals can be washed with a solvent to wash out the mother liquor.

Optionally, the isolated solid may be further dried. Drying can be carried out at reduced pressures, such as below about 200 mm Hg or below about 50 mm Hg, at temperatures such as about 35° C. to about 70° C. The drying can be carried out for any desired time period that achieves a desired purity, such as times about 1 to 20 hours, or longer. Drying may also be carried out for shorter or longer periods of time depending on the product specifications.

Ramipril obtained above can be further converted to its base addition salts by reaction with the desired base in the presence of a suitable solvent.

Ramipril obtained in the present invention contains less than about 5000 ppm, or less than about 3000 ppm, or less than about 1000 ppm of methanol, and less than about 200 ppm, or less than about 100 ppm of individual residual organic solvents.

Ramipril obtained in the present invention contains less than about 100 ppm of diisopropyl ether, less than about 35 ppm of diethyl ether, less than about 75 ppm of acetone, less than about 185 ppm of ethanol, less than about 90 ppm of methylene chloride, and less than about 40 ppm of chloroform.

The D₁₀, D₅₀, and D₉₀ values are useful ways for indicating a particle size distribution. D₉₀ refers to the value for the particle size for which at least 90 volume percent of the particles have a size smaller than the value. Likewise D₅₀ and D₁₀ refer to the values for the particle size for which 50 volume percent, and 10 volume percent, of the particles have a size smaller than the value. Methods for determining D₁₀, D₅₀, and D₉₀ include laser light diffraction, such as using equipment sold by Malvern Instruments Ltd. of Malvern, Worcestershire, United Kingdom.

Ramipril obtained according to the present invention has: a mean particle size less than about 100 μm; D₁₀ less than about 10 μm, or less than about 5 μm; D₅₀ less than about 50 μm, or less than about 40 μm; and D₉₀ less than about 400 μm, or less than about 300 μm. A Malvern instrument calculates the mean particle size and gives it as D(4,3). It is the average particle size of the powder. There is no specific lower limit for any of the D values.

Ramipril obtained according to the process described in this invention has a bulk density less than about 0.8 g/ml, or less than about 0.5 g/ml, before tapping, and a bulk density less than about 1 g/ml, or less than about 0.5 g/ml, after tapping. The bulk densities are determined using Test 616 “Bulk Density and Tapped Density,” United States Pharmacopeia 24, United States Pharmacopeial Convention, Inc., Rockville, Md., 1999, pages 1913-4.

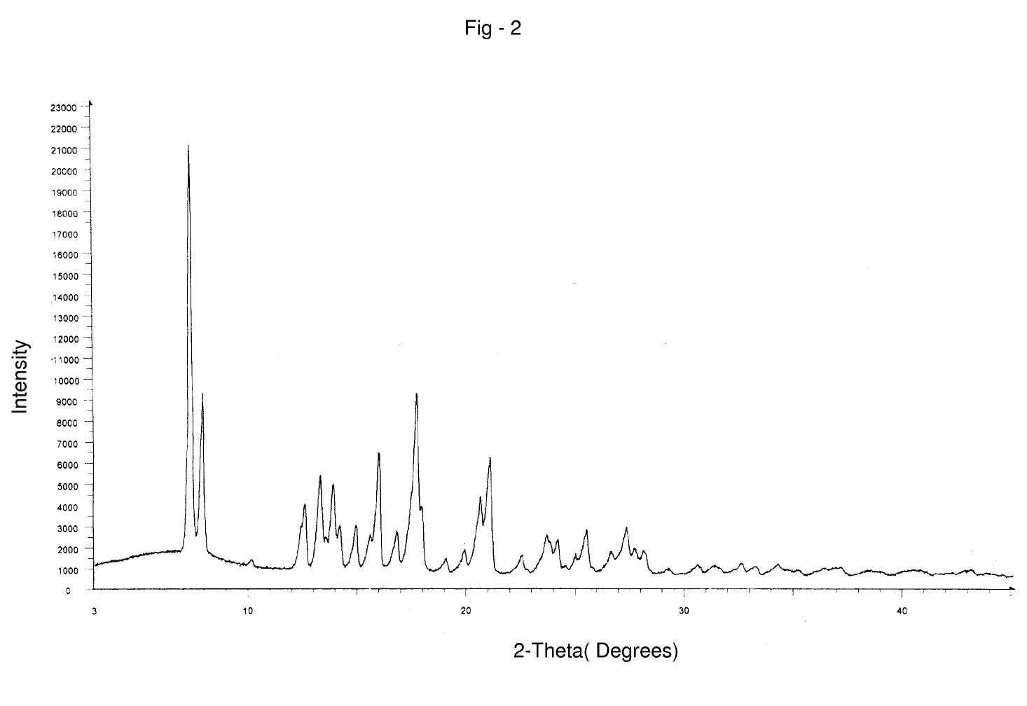

Ramipril obtained according to the process of the present invention is characterized by an XRPD pattern substantially in accordance with the pattern of FIG. 2. Ramipril obtained is also characterized by an XRPD pattern having significant peaks at about 7.3, 7.9, 12.5, 3.0, 3.8, 15.9, 17.7, 17.9, 20.6, 21.0, and 27.3, ±0.2 degrees 2θ. The pattern is also characterized by additional XRPD peaks at about 14.1 and 14.9, ±0.2 degrees 2θ

It has been observed that ramipril is a relatively unstable substance, which is susceptible to degradation at higher temperatures. The percentage of the ramipril diketopiperazine impurity increases at higher temperatures.

The susceptibility of ramipril to degradation can lead to deviation of the drug product from regulatory purity requirements, prior to the product reaching the pharmaceutical product formulation procedure. Therefore, to provide consistent purity of ramipril, packaging conditions have been developed such that they delay or prevent the formation of ramipril diketopiperazine impurity.

The packaging conditions comprise ramipril in a sealed container, under an inert atmosphere with a desiccant and an oxygen adsorbent, in a sealed triple laminated bag. The sealed pack is optionally put in a high-density polyethylene (“HDPE”) container.

The inert atmosphere can be provided using any of the inert gases such as nitrogen, argon, and the like. The gas should not react with ramipril and should be free from moisture.

The moisture adsorbent and the oxygen adsorbent are included in order to absorb any moisture and oxygen which enters the outer packaging.

Suitable moisture adsorbents which can be used in the present invention include, but are not limited to, molecular sieve zeolites, high silica zeolites having a high silica/alumina ratio of 25 or more, such as ZSM-5 (made by Mobil Oil Co., silica/alumina ratio of 400), silicalite, USY (Ultra Stable Y type zeolite, by PQ Corp., silica/alumina ratio of 78), mordenite and the like, low silica system zeolites such as Ca—X type zeolite, Na—X type zeolite, silica super fine granulated particles (for example, particle having an average particle size of 1.5 mm which has been obtained by granulating the silica super fine particle having a size of 0.1 μm or less), silica gel, γ-alumina, and the like.

Suitable oxygen adsorbents which can used include, but are not limited to CuO (that has been activated by reduction with hydrogen) on an inorganic oxide, a sachet of AGELESS™ Z 200 which reduces the oxygen concentration in a sealed container to below 0,01% creating a very low-oxygen environment. AGELESS sachets contain fine iron powder covered with sea salt and a natural zeolite impregnated with a NaCl solution and are sold by Conservation Materials Ltd., P.O. Box 2884, Sparks, Nev. U.S.A. One sachet of Ageless Z 2000 absorbs 2000 ml of oxygen (the oxygen from 10 L of air) and other similar oxygen absorbents can be used.

The above packaging and provides substantially pure ramipril, which is stable and does not undergo degradation, and also results in minimizing the ramipril diketopiperazine impurity.

Still another aspect of the present invention provides stable pharmaceutical compositions of ramipril or its pharmaceutically acceptable salts.

As discussed earlier, ACE inhibitors like ramipril, on contact with some of the commonly used pharmaceutical excipients, undergo degradation at accelerated rates.

These drugs are therefore not sufficiently stable to enable long shelf life. It is thus generally difficult to select the excipients that enable dosage forms with adequate stability.

The degradation of ramipril occurs mainly via two pathways: the hydrolysis to ramipril diacid (Formula Ie) and the cyclization to ramipril diketopiperazine (Formula Id).

An embodiment of a stable pharmaceutical composition of the present invention comprises:

a) an effective amount of ramipril or its salt, which is susceptible to cyclization, hydrolysis, and/or discoloration, and

b) an effective amount of magnesium oxide and a hydrolysis-minimizing agent suitable to retard cyclization, hydrolysis, and/or discoloration, wherein the magnesium oxide is a principal cyclization stabilizer component of the composition.

Ramipril or its pharmaceutically acceptable salts, is protected from certain forms of degradation when prepared in pharmaceutical compositions comprising magnesium oxide as the stabilizing agent.

The cyclization and hydrolytic instability which is exhibited by ramipril can be overcome via the use of a suitable quantity, i.e., an effective amount of magnesium oxide together with an agent that minimizes the hydrolysis of the ACE inhibitor, such as a saccharide. While additional stabilizers may be present in the present invention, their cyclization stabilizing effects on the ACE inhibitor formulations are minimal in comparison to the stabilizing effects of the magnesium oxide. Even small amounts of magnesium carbonate, which can result from the exposure of magnesium oxide to water and air, will have a minimal stabilizing effect on the ACE inhibitor formulations when compared to the stabilizing effect of the magnesium oxide present in the formulation.

Magnesium oxide, or calcined magnesia, is commercially available from such companies as Dead Sea Periclase of Israel, Lohmann of Germany or Morton International. This compound occurs in nature as the mineral periclase

Magnesium oxide is available in many commercial grades, all of which are within the scope of the present invention. Two forms of magnesium oxide are a very bulky form termed “Light” and a dense form termed “Heavy.” Either of the forms or combinations thereof can be used as stabilizers in the present invention.

In another embodiment the invention includes the concentration of magnesium oxide used as a stabilizer in the invention. Magnesium oxide is used in a concentration range of about 0.01% to about 5%, or about 0.04% to about 0.25%, of the total weight of the composition.

In another embodiment of the present invention the w/w ratio of ramipril or its salt to stabilizer range from about 1:1 to about 1:0.001, or from about 1:1 to about 1:0.002, or from about 1:0.2 to about 1:0.02.

The hydrolysis-minimizing agents of the present invention act to protect the ACE inhibitor from hydrolytic degradation. The hydrolysis-minimizing agent(s) to be used in the pharmaceutical products and methods of the invention are substances, which are compatible with magnesium oxide so that they do not interfere with magnesium oxide's function in the composition. Generally, they are substances, which do not contain groups significantly interfering with the function of either the metal-containing component or the drug component. Examples of useful hydrolysis-minimizing agents of the present invention are saccharides such as starch, mannitol, lactose, and other sugars that have a hydrolysis minimizing effect on the ACE inhibitors. Starches like pregelatinized starch (commercially available as PCS PC10 from Signet Chemical Corporation and Starch 1500, Starch 1500 LM grade (low moisture content grade) from Colorcon) and fully pregelatinized starch (commercially available as National 78-1551 from Essex Grain Products) are used. Different grades of lactose include but are not limited to lactose monohydrate, lactose DT (direct tabletting), lactose anhydrous, Flowlac (available from Meggle Products), Pharmatose (available from DMV), etc.

Generally, the quantity of the hydrolysis-minimizing agent present will be from about 5% to about 99%, or from about 5% to about 90%, of the total weight of the composition.

In another embodiment of the present invention, the w/w ratios of active ingredient to the hydrolysis minimizing agent range from about 1:1 to about 1:400, or from about 1:1 to about 1:200, or from about 1:10 to about 1:100.

In another embodiment, the invention includes the concentration of ramipril in the pharmaceutical composition ranging from about 1% to about 50%, or from about 1% to about 25%, or from about 1% to 12.5%, of the final composition. All the percentages stated herein are weight percentages based on total composition weight, unless otherwise stated.

The dosage forms of the pharmaceutical preparations made in accordance with the invention are solid dosage forms, which include but are not limited to capsules, tablets, caplets, pills, powders granules, etc. The optional excipients which can be used in the instant compositions are also substances which must be compatible with magnesium oxide so that they do not interfere with its function in the composition.

In solid dosage forms, the active compound and magnesium oxide are present with at least one pharmaceutical excipient.

Fillers or diluents: Various useful diluents include but are not limited to starches, lactose, mannitol, cellulose derivatives and the like. Different grades of lactose include but are not limited to lactose monohydrate, lactose DT (direct tableting), lactose anhydrous, Flowlac™ (available from Meggle products), Pharmatose™ (available from DMV) and others. Different grades of starches included but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, and tribasic calcium phosphate.

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidone, examples of commercially available crospovidone products including but not being limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcellulose. Examples of low-substituted hydroxypropylcellulose include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate(type A or type B), colloidal silicon dioxide, and starch.

Binders: Various useful binders include but are not limited to hydroxypropylcellulose (Klucel™-LF), hydroxypropyl methylcellulose (Methocel™), polyvinylpyrrolidone or povidone (PVP-K25, PVP-K29, PVP-K30, PVP-K90D), powdered acacia, gelatin, guar gum, carbomer (e.g. carbopol), methylcellulose, polymethacrylates, and starch.

Surfactants:

Various useful surfactants include but are not limited to sodium lauryl sulfate, polysorbate 80, poloxamer 188, poloxamer 407, sodium carboxy methylcellulose hydrogenated oil, polyoxyethylene glycol, and polyoxypropylene glycol, Polyoxyethylene sorbitan fatty acid esters, polyglycolized glycerides grades such as GELUCIRE 40/14, GELUCIRE 42/12, GELUCIRE 50/13 and so on.

Various glidants or anti-sticking agents which can be used include but are not limited to talc, silica derivatives, colloidal silicon dioxide.

Various solvents which can be used include but are not limited to water, lower alcohols like methanol, ethanol, and isopropanol, acidified ethanol, acetone, polyols, polyethers, oils, esters, alkyl ketones, methylene chloride, castor oil, ethylene glycol monoethyl ether, diethylene glycol monobutyl ether, diethylene glycol monoethyl ether, dimethylsulphoxide, dimethylformamide, and tetrahydrofuran.

Various pH modifiers which can be used include but are not limited to citrates, phosphates, carbonates, tartrates, fumarates, acetates, amino acid salts, and meglumine.

Various lubricants which can be used include but are not limited to magnesium stearate, sucrose esters of fatty acid, polyethylene glycol, talc, stearic acid, sodium stearyl fumarate, zinc stearate, and castor oils.

The flavoring agents, which can be used in the present invention, include but are not limited to natural or synthetic or semi-synthetic flavors like menthol, fruit flavors, citrus oils, peppermint oil, spearmint oil, and oil of wintergreen (methyl salicylate).

Various useful colorants include but are not limited to Food Yellow No. 5, Food Red No. 2, Food Blue No. 2, and the like, food lake colorants, ferric oxide, and Sunset yellow FCF.

Various film forming agents include but are not limited to cellulose derivatives such as soluble alkyl- or hydroalkyl-cellulose derivatives such as methyl cellulose, hydroxymethyl cellulose, hydroxyethyl cellulose, hydroxypropyl cellulose, hydroxymethyethyl cellulose, hydroxypropylmethyl cellulose, sodium carboxymethyl cellulose, etc., acidic cellulose derivatives such as cellulose acetate phthalate, cellulose acetate trimellitate and methylhydroxypropylcellulose phthalate, polyvinyl acetate phthalate, etc., insoluble cellulose derivatives such as ethylcellulose and the like, dextrins, starches and starch derivatives, polymers based on carbohydrates and derivatives thereof, natural gums such as gum Arabic, xanthans, alginates, polyacrylic acid, polyvinylalcohol, polyvinyl acetate, polyvinylpyrrolidone, polymethacrylates and derivatives thereof (EUDRAGIT), chitosan and derivatives thereof, shellac and derivatives thereof, and waxes and fat substances.

In the case of polymethacrylates, cationic copolymerizates of dimethylaminoethyl methacrylate with neutral methacrylic esters (EUDRAGIT™ E), copolymerizates of acrylic and methacrylic esters having a low content of quaternary ammonium groups (described in “Ammonio Methacrylate Copolymer Type A or Type B” USP/NF, EUDRAGIT™ RL and RS, respectively), and copolymerizates of ethyl acrylate and methyl methacrylate with neutral character (in the form of an aqueous dispersion, described in “Polyacrylate Dispersion 30 Per Cent” Ph. Eur., EUDRAGIT™ NE 30 D) are useful.

Anionic copolymerizates of methacrylic acid and methyl methacrylate (described in “Methacrylic Acid Copolymer, Type C” USP/NF, EUDRAGIT™ L and S, respectively, or in the form of the EUDRAGIT™ L 30 D aqueous dispersion), acidic cellulose derivatives such as cellulose acetate phthalate, cellulose acetate trimellitate and methylhydroxypropylcellulose phthalate, polyvinyl acetate phthalate, etc. may be used for film coatings.

Various plasticizers include but are not limited to castor oil, diacetylated monoglycerides, dibutyl sebacate, diethyl phthalate, glycerin, polyethylene glycol, propylene glycol, triacetin, triethyl citrate. Also mixtures of plasticizers may be utilized. The type of plasticizer depends upon the type of coating agent. A plasticizer is normally present in an amount ranging from about 5% (w/w) to about 30 (w/w) based on the total weight of the film coating.

An opacifier like titianium dioxide may also be present in an amount ranging from about 10% (w/w) to about 20% (w/w) based on the total weight of the coating. When coloured tablets are desired then the colour is normally applied in the coating. Consequently, colouring agents and pigments may be present in the film coating. Various colouring agents include but are not limited to ferric oxides, which can either be red, yellow, black or blends thereof.

Anti-adhesives are frequently used in the film coating process to avoid sticking effects during film formation and drying. An example of an anti-adhesive for this purpose is talc. The anti-adhesive and especially talc is present in the film coating in an amount of about 5% (w/w) to 15% (w/w) based upon the total weight of the coating.

Suitable polishing agents include polyethylene glycols of differing molecular weight or mixtures thereof, talc, surfactants (e.g. glycerol mono-stearate and poloxamers), fatty alcohols (e.g., stearyl alcohol, cetyl alcohol, lauryl alcohol and myristyl alcohol) and waxes (e.g., carnauba wax, candelilla wax and white wax). In an embodiment, polyethylene glycols having molecular weights of about 3,000-20,000 are employed.

In addition to above the coating ingredients, sometimes ready mixed coating materials such as those sold under the trademark OPADRY (supplied by Colorcon) may be used.

Solid compositions of a similar type may also be employed as fillers in soft and hard filled gelatin capsules using such excipients as lactose or milk sugar as well as high molecular weight polyethylene glycols and the like.

Solid dosage forms such as tablets, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions, which can be used, are polymeric substances and waxes. The active compounds can also be in microencapsulated form, if appropriate, with one or more of the above-mentioned excipients.

A further aspect of the present invention relates to a process for the preparation of the stable composition of ramipril or its pharmaceutically acceptable salt. In an embodiment, the process comprises contacting an effective amount of the drug with an effective amount of magnesium oxide and a hydrolysis-minimizing agent suitable to retard cyclization, hydrolysis, and/or discoloration, wherein the magnesium oxide is the principal cyclization stabilizer component of the composition. Any techniques known to those of skill in the art for contacting the drug and the magnesium oxide, and which are appropriate, can be employed.

In one of the embodiments, a method of preparing the pharmaceutical composition includes but is not limited to physical mixing, or blending, or dry granulation, or wet granulation.

In an embodiment, the present invention is directed to a process for preparing a stabilized pharmaceutical composition comprising an effective amount of an ACE inhibitor and a hydrolyzing agent and containing an effective amount of magnesium oxide as the cyclization stabilizing agent, wherein the process comprises:

1) Dividing a hydrolyzing agent into four different parts for geometrically mixing with the drug.

2) Blending Part I of the hydrolyzing agent, ramipril, magnesium oxide for an appropriate time.

3) Sifting step 2 through appropriate mesh sieve.

4) Sifting hydrolyzing agent Part II and the step 3 blend through an appropriate mesh sieve.

5) Sifting hydrolyzing agent Part III and Part IV separately through an appropriate mesh sieve.

6) Loading sifted hydrolyzing agent Part III, the step 4 blend and sifted hydrolyzing agent Part IV serially into a double cone blender and blending for an appropriate time.

7) Unloading from the double cone blender and filling into empty hard gelatin capsules.

A still further aspect of the present invention provides a method of using the stable pharmaceutical compositions of ramipril or its pharmaceutically acceptable salt prepared according to the process of the present invention as an antihypertensive agent.

Certain specific aspects and embodiments of this invention are described in further detail by the examples below, which examples are provided only for the purpose of illustration and are not intended to limit the scope of the appended claims in any manner.

### EXAMPLE 1

### Preparation of the S,S,S-Diasteromeric Salt of 2-Azabicyclo[3,3,0]-Octane-3-Carboxylic Acid Benzyl Ester (Formula III)

100 g of 2-Azabicyclo[3,3,0]-octane-3-carboxylic acid benzyl ester hydrochloride of Formula II was taken into a clean and dry 4 neck round bottom flask containing 400 ml of ethyl acetate. The mixture was stirred for 10 minutes. A solution of 25.6 g of sodium hydroxide in 128 ml of water was added slowly to the above mixture. The organic layer was separated. The aqueous layer was extracted into 200 ml of ethyl acetate. The combined organic layer was filtered through a celite bed, and the bed was washed with 100 ml of ethyl acetate. The combined organic layer was distilled at 50° C. under a vacuum of 300 mm Hg to give a residue.

54 g of L-(+)-mandelic acid was taken into a clean and dry round bottom flask containing 350 ml of ethyl acetate and stirred for 10 minutes. The residue obtained above was added slowly to the solution of madelic acid at 28° C. and then the reaction mass was cooled to 0° C. The reaction mass was maintained at 0° C. for 1 hour. The separated solid was filtered and washed with 50 ml of ethyl acetate. The wet solid was dried at 30° C. under a vacuum of 350 mm Hg to afford 70.52 g of the title compound.

Purity by HPLC: 99.5%.

### EXAMPLE 2

### Preparation of the S,S,S-Diasteromeric Salt of 2-Azabicyclo[3,3,0]-Octane-3-Carboxylic Acid Benzyl Ester (Formula III)

100 kg of 2-Azabicyclo[3,3,0]-octane-3-carboxylic acid benzyl ester hydrochloride of Formula II was take into a reactor, 400 liters of ethyl acetate was added and the mixture was stirred for 10 minutes. A solution of 25.6 kg of sodium hydroxide flakes in 128 liters of water was added to the above mixture slowly. The reaction mixture was checked for clear dissolution. After clear dissolution was obtained, the reaction mass was allowed to settle for 20 minutes, and the organic layer was separated. The aqueous layer was extracted into 200 liters of ethyl acetate, followed by extraction into 200 liters of ethyl acetate in two equal portions. The organic layer was distilled completely below a temperature of 57° C. under a vacuum of 600 mm Hg to get a residue. The residue was added to a solution of 53.94 kg of mandelic acid in 350 liters of ethyl acetate at 22° C. The reaction mass was stirred for 15 minutes and observed for solid separation. Then the reaction mass was cooled to 2° C. and maintained for 1 hour. The separated solid was filtered in a centrifuge and washed with 80 liters of ethyl acetate in 5 equal lots. The wet material was air dried at 25° C. for 4 hours to yield 64.5 kg of the title compound.

### EXAMPLE 3

### Preparation of (S,S,S)-Azabicyclo[3,3,0]-Octane3-Carboxylic Acid Benzyl Ester Hydrochloride (Formula IV)

55 g of the S,S,S -diastereomeric salt of 2-azabicyclo[3,3,0]-octane-carboxylic acid benzyl ester of Formula III was taken into a round bottom flask containing 450 ml of dichloromethane. The mixture was stirred for 10 minutes at 25° C. A solution of 11.2 g of sodium hydroxide in 45 ml of water was added to the above reaction mass at 0-3° C. The reaction mixture was maintained at 0-3° C. for 15 minutes, and then the organic layer was separated. The resultant aqueous layer was washed with 165 ml of dichloromethane in three equal lots. The pH of the aqueous layer was adjusted to 2.5 using 15.4 ml of aqueous hydrochloric acid. The resultant reaction solution was stirred for about 3 hours at 0-3° C. The separated solid was filtered and washed with 110 ml of dichloromethane, and the wet solid was dried at 65° C. under a vacuum of 350 mm Hg for 7 hours to afford 36.2 g of title compound.

Purity by HPLC: 100%.

### EXAMPLE 4

### Preparation of (S,S,S)-Azabicyclo[3,3,0]-Octane-3-Carboxylic Acid Benzyl Ester Hydrochloride (Formula IV)

64.5 kg of the compound obtained in Example 2 was added to a reactor containing 322 liters of dichloromethane. The mixture was stirred for 10 minutes and then cooled to 2° C. A solution of 13.12 kg of sodium hydroxide flakes in 53.8 liters of water was added to the above mixture at 2 to 3° C. and the reaction mass was stirred at the same temperature for one hour. The organic layer was separated and the aqueous layer was extracted into 3×64.5 liters of dichloromethane. The combined organic layer was taken into another reactor and 15.7 liters of 36% aqueous hydrochloric acid was added at 2 to 3° C. The reaction mass was maintained at 2 to 3° C. for 2 hours. The separated solid was filtered and washed with 64.5 liters of chilled dichloromethane. The wet solid was dried at 62° C. for 2 hours to yield 43 kg of the title compound.

Purity by HPLC: 99.93%.

### EXAMPLE 5

### Preparation of 1-[N-(1-(S)-Carboethoxy-3-Phenyl Propyl)-(S)-Alanyl]-Cis-Endo Octahydrocyclopenta[b ]Pyrrole-2-Carboxylic Acid BENZYL ESTER (FORMULA VI)

### Step a): Preparation of ECPP alanine acid chloride of Formula V

26.9 g ECPP alanine (ethoxycarbonyl phenyl propyl alanine), 243 ml of toluene and 27 ml of chloroform were taken into a clean dry round bottom flask. The resultant mass was cooled to 15° C. followed by passing dry hydrogen chloride gas through the mass for 15 minutes, until the mass pH was below 2. The resultant suspension was further cooled to 0° C. followed by addition of 24.8 g of phosphorous pentachloride. The reaction mixture was stirred for 60 minutes and allowed to reach 25° C. The suspension was stirred for 90 minutes followed by filtration to separate the solid. The separated solid was washed with 27 ml of toluene and 75 ml of petroleum ether to afford 28.57 g of ethoxycarbonyl phenylpropyl alanoyl chloride hydrochloride of Formula VI.

### Step b): Preparation of 1-[N-(1-(S)-carboethoxy-3-phenyl propyl)-(S)-alanyl]-cis-endo octahydro cyclopenta[b]pyrrole-2-carboxylic acid benzyl ester of Formula VI

18.2 g of (S,S,S)-azabicyclo[3,3,0]-octane-3-carboxylic acid benzyl ester hydrochloride of Formula IV and 300 ml of dichloromethane were taken into a clean and dry round bottom flask followed by stirring. The resultant mass was cooled to 10° C. followed by addition of 22.5 g of triethylamine. 28.7 g of ECPP alanine acid chloride of Formula V was dissolved in 515 ml of dichloromethane and the solution was added slowly to the above mass at 0 to 3° C. The resultant mixture was stirred for 18 hours at 0 to 3° C. under a nitrogen atmosphere with HPLC monitoring for reaction completion. After completion of the reaction, 490 ml of water was added to the reaction mixture and stirred for 15 minutes. The organic layer was separated and washed with a solution of 13.7 g of sodium carbonate in 275 ml of water, followed by washing with a solution of 27.5 ml of hydrochloric acid in 275 ml of water. The acid-base washing was repeated twice with the same quantities and then the organic layer was finally washed with 1960 ml of water in 4 equal lots. The resultant organic layer was distilled completely under vacuum at about 30° C. to afford 37.5 g of the title compound.

Purity by HPLC: 92-94%.

### EXAMPLE 6

### Preparation of 1-[N-(1-(S)-Carboethoxy-3-Phenyl Propyl)-(S)-Alanyl]-Cis-Endo Octa Hydro Cyclo Penta-[b]Pyrrole-2-Carboxylic Acid BENZYL ESTER (FORMULA II)

405 liters of toluene was taken into a reactor and 45 liters of chloroform was added to it. the mixture was cooled to 25°C. 45 kg of ECCP alanine was added to the mixture at 24° C. and the mass was further cooled to 15°C. HCl gas was passed into the mass for 30 minutes, until the mass pH was below 2, and the mass was cooled to 3° C. 49.6 kg of PCl₅ was then added to the mass at 8° C. The mass was maintained at 5° C. for 1 hour, then the mass temperature was raised to 25° C. and maintained for 2 hours. The mass was then filtered and the filter bed was washed with 45 liters of toluene. The solid was then washed with 135 liters of petroleum ether. The wet solid material was dried at 25° C. under a nitrogen pressure of 0.8 kg/cm².

875 liters of dichloromethane, and 35 kg of (S,S,S)-azabicyclo[3,3,0]-octane-3-carboxylic acid benzyl ester hydrochloride of Formula IV were taken into a reactor and stirred for 15 minutes. 98 liters of triethylamine was added to the mass and stirred for 25 minutes. The mass was then cooled to 2.5° C. and a solution of 58 kg of ECPP alanine acid chloride of Formula V in 850 liters of dichloromethane was added at 2 to 3° C. under a nitrogen atmosphere. The mass was maintained at 2 to 3° C. for 10 hours. Reaction completion was checked using thin layer chromatography. After the reaction was completed, 820 liters of water was added to the mass and stirred for 15 minutes. The organic layer was separated and washed with a solution of 46 kg of sodium carbonate in 910 liters of water in two equal lots. The organic layer was then washed with 480 liters of water. Then the organic layer was washed with a solution of 138 liters of 36% aqueous hydrochloric acid in 1365 liters of water in three equal lots. Finally the organic layer was washed with 1920 liters of water in four equal lots. The organic layer was distilled completely below 35° C. under a vacuum of 550 mm Hg to get 70 kg of the title compound.

### EXAMPLE 7

### Preparation of 1-[N-(1-(S)-Carboethoxy-3-Phenyl Propyl)-(S)-Alanyl]-Cis-Endo Octa Hydro Cyclo Penta Pyrrole-2-Carboxylic Acid (FORMULA I)

36 g of 1-[N-(1-(S)-Carboethoxy-3-phenyl propyl)-(S)-Alanyl]-cis-endo octa hydro cyclo penta[b]Pyrrole-2-carboxylic acid benzyl ester was taken into an autoclave containing 200 ml of ethanol. 11.6 g of wet palladium on carbon [10% Pd]was added to the mixture and maintained at 2.5-3 kg/cm² hydrogen pressure at 10-15° C. for 12.5 hours. Reaction progress was checked using HPLC. After the completion of the reaction the reaction mixture was filtered through a celite bed and the celite bed was washed with 50 ml of acetone. The resultant filtrate was distilled completely at about 15-20° C. under a vacuum of about 350-400 mm Hg to afford a crude title compound.

The above-obtained crude compound was dissolved in a mixture of 65 ml of diisopropyl ether and 33 ml of diethyl ether followed by cooling to 0-5° C. The mass was maintained at 0 to 5° C. for 60 minutes. The solid was separated by filtration and washed with a mixture of diisopropyl ether and diethyl ether 33 ml:17 ml, to obtain 29.6 g of the title compound.

### EXAMPLE 8

### Preparation 0f 1-[N-(1-(S)-Carboethoxy-3-Phenyl Propyl)-(S)-Alanyl]-Cis-Endo Octa Hydro Cyclo Penta Pyrrole-2-Carboxylic Acid (FORMULA I)

613 liters of ethanol was taken into a reactor. A solution of 70 liters methanol and 102 kg of 1-[N-(1-(S)-Carboethoxy-3-phenyl propyl)-(S)-Alanyl]-cis-endo octa hydro cyclo penta[b]Pyrrole-2-carboxylic acid benzyl ester was prepared and added to the ethanol. 15.74 kg of palladium on carbon (50% wet) was added to the above reaction mass. The reaction mass was cooled to 11° C. and a hydrogen pressure of 3 kg/cm² was applied. The reaction mass was maintained at this pressure for 2 hours and the reaction progress was monitored using thin layer chromatography. After the reaction was completed, the mass was filtered through a Hyflow bed and the bed was washed with 175 liters of acetone. Again the filtrate was filtered through a fresh Hyflow bed and the bed was washed with 70 liters of acetone. The combined filtrate was distilled completely at 13 to 16° C., under a vacuum of 700 mm Hg. In a separate reactor, a mixture of 182 liters of diisopropyl ether and 91 liters of diethyl ether was prepared and this mixture was added to the residue obtained after distillation. The suspension was then cooled to 2° C. and maintained for 1 hour. The solid was separated by filtration and washed with a chilled mixture of 98 liters of diisopropyl ether and 49 liters of diethyl ether. The wet material was dried at 28° C. for 6 hours to yield 64.8 kg of the title compound.

Purity by HPLC: 99.8%.

Individual process related impurities: less than 0.15%.

Residual solvents: Diisopropyl ether less than 29 ppm; Diethyl ether less than 19 ppm; Acetone less than 16 ppm.

Particle Size Distribution: D₁₀ 4 μm, D₉₀ 54 μm.

Bulk Density: Before tapping 0.21 g/ml.
After tapping 0.37 g/ml.

### EXAMPLE 9

### Purification of 1-[N-(1-(S)-Carboethoxy-3-Phenyl Propyl)-(S)-Alanyl]-Cis-Endo Octa Hydro Cyclo Penta Pyrrole-2-Carboxylic Acid (Formula I)

3.47 liters of diisopropyl ether and 1.73 liters of diethyl ether are taken into a reactor and 1.0 kg of ramipril is added. The mixture is stirred for about 45 minutes at about 28° C. 2.27 liters of water is then added and stirred for 45 minutes at about 28° C. The mass is then further cooled to 0° C. and maintained for 1 hour. The separated solid is filtered and washed with a mixture of 0.2 liters of diisopropyl ether and 0.2 liters of diethyl ether. The wet material is dried at 45° C. for 6 hours.

In another reactor, 2.5 liters of diisopropyl ether and 1.5 liters of acetone is taken and the dry material obtained above is added to it. The mixture is stirred for 45 minutes at about 28° C., and then cooled to 0° C. and maintained further for 60 minutes. The solid is separated by filtration and washed with 0.5 liters of a 1:1 mixture of chilled diisopropyl ether and acetone. The wet material is dried at 50° C. for 6 hours to yield 0.8 kg of the title compound.

### EXAMPLE 10

### Stabilization of Ramipril (Packaging)

The compound was placed in a clean polyethylene bag, which was tied and placed into a black polyethylene bag along with a silica gel pouch and an antioxidant. The black bag was flushed with nitrogen, and then sealed. The black bag was placed in a high-density polyethylene drum along with a silica gel pouch, then was flushed with nitrogen and finally the drum was sealed.

Storage stability studies were done at the following different conditions: at temperature of 2-8° C.; at 25±2° C. and 60±5% relative humidity (“RH”), and at 40 ±2° C. and 75±5% RH. There was no change in the impurity content in packaged ramipril under these storage conditions up to about 48 months.

### EXAMPLE 11

### Determination of Impurities in Ramipril

The analysis of conditions for determining the level of impurities in ramipril using HPLC are described in the table.

All the impurities are tested according to an HPLC method performed using an Inertsil ODS 3V column (250×4.6 mm, 5 μm or Lichrospher, 100, RP-18e, 250×4.0 mm, 5 μm) with the following parameters:

| Flow rate | 1.0 ml/minute | | |
|---|---|---|---|
| Detector | 210 nm | | |
| Injection load | 10 μl | | |
| Temperature | 65° C. | | |
| | | | Mobile |
| Gradient | | Mobile phase | phase B |
| conditions | Interval (min) | A (percent v/v) | (percent v/v) |
| | 0.00 | 90 | 10 |
| | 7.00 | 80 | 20 |
| | 19.00 | 50 | 50 |
| | 30.00 | 35 | 65 |
| | 50.00 | 25 | 75 |
| | 55.00 | 90 | 10 |
| | 60.00 | 90 | 19 |

| | |
|---|---|
| Mobile phase | Mobile phase A: Dissolve 2 g of sodium perchlorate in a |
| | mixture of 0.5 ml of triethyl amine and 800 ml of water; |
| | adjust to pH 3.6 ± 0.1 with phosphoric acid and add 200 |
| | ml of acetonitrile |
| | Mobile phase B: Dissolve 2 g of sodium perchlorate in a |
| | mixture of 0.5 ml of triethyl amine and 300 ml of water; |
| | adjust to pH 2.6 ± with phosphoric acid and add 700 ml |
| | of acetonitrile |

| | Relative |
|---|---|
| | Retention Time |
| Compound | (minutes) |
| Ramipril methyl ester impurity | 0.85 |
| Ramipril isopropyl ester | 1.15 |
| Hexahydro ramipril | 1.32 |
| Ramipril diketopiperazine | 1.5 |
| Ramipril diacid | 0.34 |
| ECPP Alanine | 0.4 |
| Ramipril dimer | 2.36 |
| Toluene | 1.4 |
| Ramipril | 1.0 |

Relative retention times are calculated by dividing retention time for a peak by the retention time for ramipril.

For determining the chiral purity and residual solvents, European Pharmacopeal methods are used, as given in European Pharmacopoeia 5.0, Volume 2, Pages 2355-2357.

### EXAMPLES 12-16

PREPARATION OF RAMIPRIL CAPSULES

| | Comparative | Example | Example | Example | Example | Example |
|---|---|---|---|---|---|---|
| | Example | 12 | 13 | 14 | 15 | 16 |
| Component | mg/Capsule | | | | | |
| (Strength) | 1.25 mg | 1.25 mg | 1.25 mg | 2.5 mg | 5.0 mg | 10 mg |
| Ramipril | 1.25 | 1.25 | 1.25 | 2.5 | 5 | 10 |
| Pregelatinized | — | — | 106.7 | 105.6 | 103.32 | 98.8 |
| Starch (PCS | | | | | | |
| PC10) | | | | | | |
| Pregelatinized | — | — | 11.85 | 11.7 | 11.48 | 11 |
| Starch (78- | | | | | | |
| 1551) | | | | | | |
| Pregelatinized | 118.75 | 118.2 | — | — | — | — |
| Starch | | | | | | |
| (Starch 1500 | | | | | | |
| M) | | | | | | |
| Magnesium | — | | 0.2 | 0.2 | 0.2 | 0.2 |
| Oxide (Light) | | | | | | |
| Magnesium | — | 0.26 | — | — | — | — |
| Oxide (heavy) | | | | | | |

| | |
|---|---|
| Fill weight | 120 mg |

Manufacturing Procedure:

1) Pregelatinized starch (PCS PC 0) is divided into four different parts for geometrically mixing with the drug.

2) Loaded into double cone blender in the following sequence:
a) Pregelatinized Starch (PCS-PC10) Part I.
b) Ramipril.
c) Magnesium oxide.
d) Fully pregelatinized starch (National 78-1551).
Mixed for 10 minutes.

3) Sifted material of Step 2 through a 30 mesh sieve.

4) Sifted together pregelatinized Starch (PCS-PC10) Part II and material of Step 3 through a 30 mesh sieve.

5) Sifted pregelatinized Starch (PCS-PC10) Part III through a 30 mesh sieve.

6) Sifted pregelatinized Starch (PCS-PC10) Part IV through a 30 mesh sieve.

7) Loaded into double cone blender in the following sequence:
a) Material of Step 5.
b) Material of Step 4.
c) Material of Step 6.
Mixed for 20 minutes

8) Unloaded from double cone blender.

### EXAMPLE 17

Stability of capsules prepared in Example 12 and the comparative example (“CE”) was tested at 40° C. and 75% RH for 3 months. Packaging used was HDPE containers. The data is tabulated below in Table 1 for ramipril diketopiperazine impurity (“RDK”), ramipril diacid impurity (“RDA”), and total degradation products (“TDP”). The data show that the use of magnesium oxide effectively stabilizes ACE inhibitor-containing compositions when compared to similar formulations that do not contain a stabilizer.

TABLE 1

| | RDK (%) | | RDA (%) | | TDP (%) | |
|---|---|---|---|---|---|---|
| Time | Example | | Example | | Example | |
| Point | 12 | CE | 12 | CE | 12 | CE |
| Initial | 0.04 | 0.08 | 0.05 | 0.02 | 0.28 | 0.52 |
| 2 | 0.14 | 4.47 | 0.53 | 0.3 | 0.75 | 4.9 |
| months | | | | | | |
| 3 | 0.16 | 5.56 | 0.64 | 0.28 | 0.92 | 6 |
| months | | | | | | |

### EXAMPLE 18

Stability of capsules prepared in Examples 13 and 16 was tested by storage at 40° C. and 75% RH for 3 months. Packaging was in HDPE containers. The data is tabulated below in Table 2 for ramipril diketopiperazine impurity (“RDK”), ramipril diacid impurity (“RDA”) and total degradation products (“TDP”).

TABLE 2

| | Example 13 | | | Example 16 | | |
|---|---|---|---|---|---|---|
| | | RDA | TDP | | RDA | TDP |
| Time Point | RDK (%) | (%) | (%) | RDK (%) | (%) | (%) |
| Initial | 0.2 | 0.076 | 0.276 | 0.09 | 0.017 | 0.197 |
| 1 month | 0.49 | 0.19 | 0.68 | 0.38 | 0.06 | 0.46 |
| 2 months | 0.62 | 0.31 | 0.93 | 0.51 | 0.15 | 0.67 |
| 3 months | 0.37 | 0.14 | 0.51 | 0.53 | 0.22 | 0.76 |

### EXAMPLES 19 AND 20

Examples 19 and 20 are ramipril capsule compositions with different concentrations of magnesium oxide as a stabilizer.

| | |
|---|---|
| Example 19 | Example 20 |

| | |
|---|---|
| Component | mg/Capsule |

| | | |
|---|---|---|
| Ramipril | 1.25 | 1.25 |
| Pregelatinized Starch | 118.025 | 118.7 |
| (Starch 1500LM) | | |
| Magnesium Oxide (Heavy) | 0.125 | 0.05 |
| Fill weight of capsule (mg) | 119.4 | 120 |

Manufacturing procedure: The composition was prepared in the same manner as described in Example 12.

Stability of capsules prepared in Examples 12, Examples 19 and Example 20 were tested by storage at 40° C. and 75% RH for 3 months. Packaging was in HDPE containers. The data is tabulated below in Table 3 wherein ramipril diketopiperazine impurity (“RDK”), ramipril diacid impurity (“RDA”), and total degradation products (“TDP”) concentrations are expressed as weight percentages.

TABLE 3

| Time | Degradation | | | |
|---|---|---|---|---|
| Point | Product | Example 12 | Example 19 | Example 20 |
| Initial | RDK | 0.04 | 0.14 | 0.15 |
| | RDA | 0.05 | 0.07 | 0.07 |
| | TDP | 0.28 | 0.30 | 0.29 |
| 1 month | RDK | — | 0.57 | 0.95 |
| | RDA | — | 0.12 | 0.10 |
| | TDP | — | 0.8 | 1.17 |
| 2 months | RDK | 0.14 | 0.92 | 1.4 |
| | RDA | 0.53 | 0.17 | 0.21 |
| | TDP | 0.75 | 1.34 | 1.73 |
| 3 months | RDK | 0.16 | 0.86 | 1.46 |
| | RDA | 0.64 | 0.27 | 0.27 |
| | TDP | 0.92 | 1.24 | 1.93 |

### EXAMPLE 21

Ramipril 2.5 mg Tablets (aqueous granulation)

| Component | mg/Capsule |
|---|---|
| Ramipril | 2.5 |
| Lactose monohydrate | 30 |
| Pregelatinized Starch | 73 |
| Magnesium Oxide (Light) | 0.2 |
| Hydroxypropyl | 3.6 |
| methylcellulose | |
| Water | qs |
| Lactose DT | 10 |
| Iron oxide yellow | 0.2 |
| Sodium stearyl fumarate | 0.5 |

Manufacturing Procedure:

1. Mix geometrically ramipril, magnesium oxide, pregelatinized starch and lactose monohydrate.

2. Sift step 1 through a 40 mesh sieve.

3. Dissolve hydroxypropyl methylcellulose in water.

4. Granulate step 2 using binder from step 3.

5. Dry the wet mass of granules.

6. Screen the dried granules through a 30 mesh sieve.

7. Sift lactose DT through a 40 mesh sieve.

8. Sift iron oxide yellow through a 100 mesh sieve.

9. Sift sodium stearyl fumarate through an 80 mesh sieve.

10. Mix step 7 and step 8 and add to step 6.

11. Blend step 9 in blender for 25 minutes.

12. Add step 9 to step 11 and blend for 5 minutes.

13. Compress the final blend into tablets.

We claim:

1. A process for preparing ramipril or a salt thereof, comprising:
a) providing a solution of ramipril in a solvent comprising an ether, a ketone, water, or a mixture of any two or more thereof; and
b) crystallizing a solid from the solution.

2. The process of claim 1, wherein a solvent comprises a combination of an ether, a ketone, and water.

3. The process of claim 1, wherein an ether comprises diisopropyl ether

4. The process of claim 1, wherein a ketone comprises acetone.

5. The process of claim 1, wherein a solvent comprises diisopropyl ether, acetone, and water.

6. The process of claim 1, wherein recovered ramipril contains less than about 0.5 weight percent of each of the following:
a) a compound having a formula: 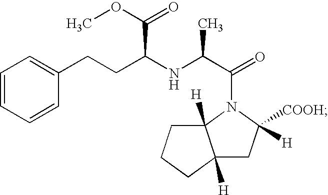
b) a compound having a formula: 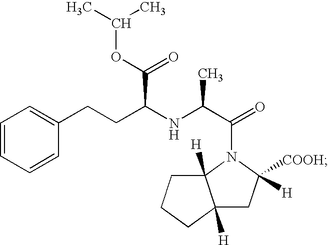
c) a compound having a formula: 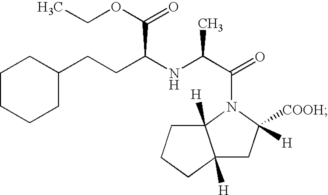
d) a compound having a formula: 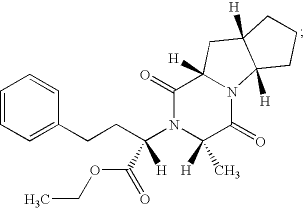
e) a compound having a formula: 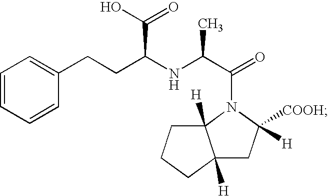
f) a compound having a formula: 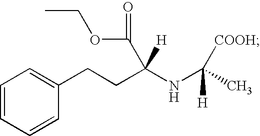
and
g) a compound having a formula:

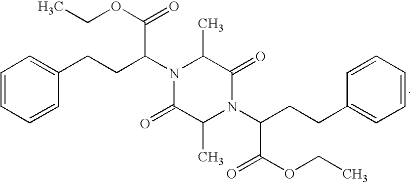

7. The process of claim 1, wherein recovered ramipril contains less than about 0.1 percent by weight of each compound of a)-g).

8. The process of claim 1, wherein recovered ramipril has a mean particle size of less than about 50 μm, D₁₀ less than about 10 μm or less than 5 μm, D₅₀ less than about 50 μm or less than 25 μm, and D₉₀ less than about 200 μm or less than 100 μm.

9. The process of claim 1, wherein recovered ramipril has a bulk density less than 0.8 g/ml, or less than 0.5 g/ml, before tapping, and a bulk density less than 1 g/ml, or less than 0.5 g/ml, after tapping.

10. A process for preparing ramipril or a salt thereof, comprising:
a) reacting a compound having Formula Ha with an optically pure acid in the presence of an alkyl acetate solvent to afford a diastereomeric salt; 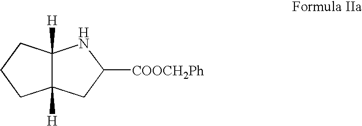
b) reacting the diastereomeric salt with a base to give a free base having Formula IV, converting a free base to its acid salt, and optionally isolating an acid salt; 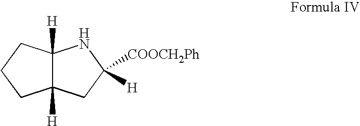
c) reacting an acid salt of b) with a compound having Formula V in the presence of a base, to afford a compound having Formula VI; and 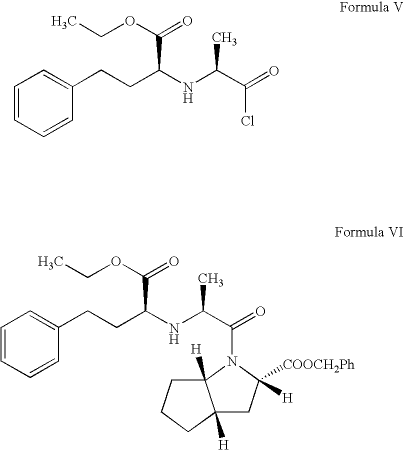
d) reacting a compound having Formula VI with a reducing agent to afford ramipril.

11. The process of claim 10, wherein an alkyl acetate solvent comprises ethyl acetate and an optically pure acid comprises a mandelic acid.

12. A method for packaging ramipril or a salt thereof, comprising:
placing ramipril in a sealed container under an inert atmosphere;
placing the sealed container, a desiccant, and an oxygen adsorbent in a second sealed container;
placing the second sealed container in a triple laminated bag and sealing; and enclosing the triple laminated bag in a closed high density polyethylene (“HDPE”) container.

13. A solid pharmaceutical composition comprising ramipril or a salt thereof, at least one pharmaceutical excipient, and about 0.01% to about 0.5% by weight of magnesium oxide.

14. The pharmaceutical composition of claim 13, comprising about 0.04% to about 0.25% by weight of magnesium oxide.

15. The pharmaceutical composition of claim 13, wherein a pharmaceutical excipient comprises a hydrolysis-minimizing agent.

16. The pharmaceutical composition of claim 13, wherein a pharmaceutical excipient comprises a saccharide.
